(19) United States
(12) Patent Application Publication
(10) Pub. No.: US 2009/0012465 A1
(43) Pub. Date: Jan. 8, 2009

(54) Microcatherer having tip relief region

(76) Inventor: Lucas Latini, Norcross, GA (US)

Correspondence Address: LAW OFFICE OF COLLEN A. BEARD, LLC, P. O. BOX 1064, DECATUR, GA, 30031-1064, US

(21) Appl. No.: 12/152,170

(22) Filed: May 13, 2008

Related U.S. Application Data
(62) Division of application No. 10/406,647, filed on Mar. 19, 2003.

(51) Int. Cl. A61M 25/00

(52) U.S. Cl. 604/82

(57) ABSTRACT

A microcatheter having a distal end with a tip relief region comprising at least one spiral cut, preferably a full thickness cut, and preferably having decreasing pitch towards the distal tip. The microcatheter is preferably made of medical grade superelastic nitinol. The microcatheter can be used alone or as one catheter in a dual lumen microcatheter assembly.

### RELATED APPLICATIONS

This application is related to U.S. Provisional application 60/365,946 filed on Mar. 19, 2002 and U.S. Provisional application 60/60/370,361 filed on Apr. 5, 2002.

### BACKGROUND OF THE INVENTION

The invention relates to a microcatheter having a distal tip with flexibility and strength. The microcatheter can be used as one catheter in a coaxial dual lumen microcatheter assembly.

A wide variety of microcatheters have been developed for insertion in the vascular system for a number of diagnostic or therapeutic applications. However, catheters that have been developed and are appropriate for use in the peripheral vasculature are typically not appropriate for use in the cranial vasculature, and other areas requiring a small diameter catheter. Such applications require a small diameter and very flexible catheter, to access small tortuous vessels.

Microcatheters having sufficient flexibility and size for use in small tortuous vessels have been developed. However, dual lumen microcatheters suitable for delivering viscous fluids to neurovascular sites have not been developed. Such dual lumen microcatheters would have a number of applications in diagnostic and interventional medicine, such as drug delivery, imaging, treatment of tumors, aneurysms, arteriovenous malformations (AVMs), etc.

Hydrogels are useful for a number of biomedical applications. Prepolymers that form hydrogels in situ are administered to the body in liquid form, whereupon they transform into the solid hydrogel. In situ forming hydrogels are especially useful for some applications, such as embolotherapy, tissue bulking, and drug delivery. In situ forming hydrogels are of several types. One type of in situ forming hydrogels is made from crosslinking prepolymers. Such prepolymers contain crosslinkable groups that can be crosslinked after administration (in situ) to form the hydrogel. See WO 01/68720 to BioCure, Inc. and U.S. Pat. No. 5,410,016 to Hubbell et al. for examples of such prepolymers.

WO 01/68720 describes a two part prepolymer system used to form a hydrogel in situ. Each of the two parts includes one part of a redox couple. When the two parts are combined, crosslinking (formation of the hydrogel) begins. It is sometimes preferable to begin this crosslinking at the intended site of application. In this case, the two parts are not combined until they are applied to the intended site. Premature mixing of the two parts can lead to unintended, premature formation of the hydrogel (and clogging of the catheter, for example).

In one embodiment disclosed in WO 01/68720, a side-by-side dual lumen catheter is used to deliver the prepolymer compositions. One lumen delivers the reducing solution and the second lumen delivers the oxidizing solution. The prepolymer can be included in one or both of the reducing and oxidizing solutions. This catheter works well for many applications. However, a disadvantage of side-by-side dual lumen catheters for use in delivering a viscous fluid is that they are generally restricted in terms of size. They cannot be made below a certain diameter and maintain the needed flexibility to access tortuous or otherwise hard to reach sites, such as, particularly, neurovascular sites- and be able to deliver a viscous fluid.

Microcatheters are needed to access many neurovascular sites and to provide super selective embolization. However, as discussed above, it has proved very difficult to design and manufacture a suitable dual lumen microcatheter.

### SUMMARY OF THE INVENTION

The invention relates to a microcatheter having a distal end with a tip relief region comprising at least one spiral cut, preferably a full thickness cut, and preferably having decreasing pitch towards the distal tip. The microcatheter is preferably made of medical grade superelastic nitinol. The microcatheter can be used alone or as one catheter in a dual lumen microcatheter assembly. The assembly is formed by inserting the microcatheter of the invention through a larger inner diameter microcatheter to form a coaxial dual lumen catheter assembly.

### BRIEF DESCRIPTION OF THE DRAWINGS

FIG. 1 is a view of the microcatheter according to the present invention.

FIG. 2 is a view of one embodiment of the tip relief region of the microcatheter according to the present invention.

FIG. 3 is a cross-sectional view of a dual lumen microcatheter assembly including the microcatheter of the present invention.

FIG. 4 is a view of one embodiment of a dual lumen microcatheter assembly including the microcatheter of the present invention.

### DETAILED DESCRIPTION OF THE INVENTION

“Microcatheter” means a catheter having a distal tip size of about 4 French or smaller.

“Strength” means both the ability of a catheter to resist fluid pressures applied to the lumen of the catheter without bursting or leaking, and the ability to resist tensile forces without tearing.

“Flexibility” means the ability of a catheter to bend when a force is applied in a direction other than along an axis of the catheter. The flexibility is inversely related to the amount of force required to deflect the catheter from an initial position.

### I. The Microcatheter

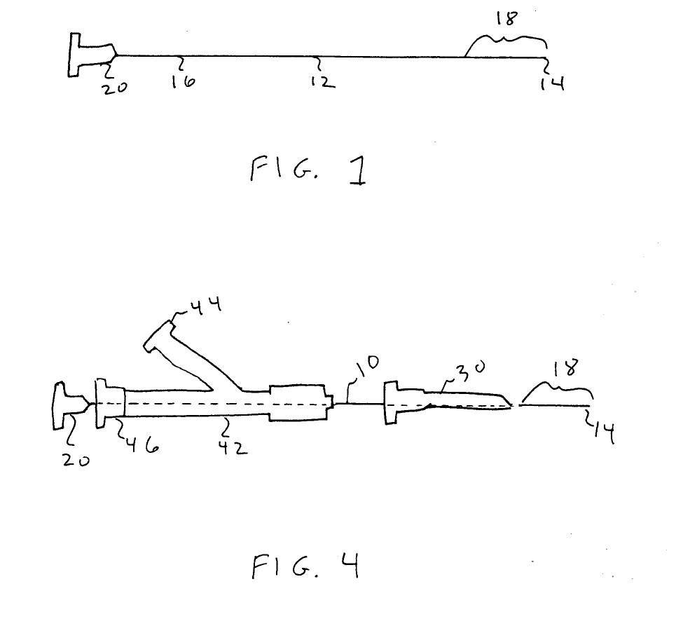

The microcatheter 10 is shown in FIG. 1 and includes an elongate tubular body 12, a distal portion 14, and a proximal portion 16. The distal portion includes a tip relief region 18. An adaptor 20 is attached to the proximal tip.

The microcatheter is designed to be used on its own, as a single lumen microcatheter, or in combination with a larger diameter microcatheter to form a coaxial dual lumen microcatheter assembly. The microcatheter of the invention is termed the “microcatheter” herein. The larger diameter microcatheter is termed the “second catheter” herein. Requirements for the larger diameter catheter can be discerned from the following discussion.

If the microcatheter is to be used as a stand alone microcatheter, its size is important only so far as it should be of an appropriate size to access the area of interest. The microcatheter can be as large as about 4 Fr. It should be understood that the microcatheter and the microcatheter assembly can also be made larger than 4 Fr. However, the microcatheter and the assembly have been specifically designed, and the design is particularly advantageous, when used as a microcatheter, i.e. having a diameter of about 4 Fr or smaller. The design is even more advantageous when a microcatheter, or dual lumen microcatheter, below about 2.8 Fr is needed. The microcatheter may be as small as about 0.7 Fr.

The microcatheter is formed from a tube desirably made of a metal, such as platinum, a platinum alloy, a nickel alloy, a titanium alloy, and some types of stainless steel (such as 316L stainless steel). Desirably, a binary nickel titanium alloy (nitinol) is used. Some plastics such as polyimide, polyethylene, polyurethane, and PTFE may be used. The requirements for the fabrication material will depend upon the desired characteristics of the microcatheter, such as flexibility and strength, and the design parameters such as length and diameter. Desirably, medical grade superelastic nitinol is used.

To provide flexibility at the distal tip, and allow the microcatheter to track, the distal tip of the microcatheter has a tip relief portion, illustrated in FIG. 2. This region is designed to provide flexibility to the distal portion of the microcatheter while maintaining strength.

The tip relief portion is provided by a region of spiral cuts at the distal tip. Desirably the cuts are full thickness. The relief region can be one continuous spiral cut or a plurality of spiral cut regions.

The spiral cuts desirably have variable pitch (distance between cuts—expressed as cuts per millimeter (C/mm)). Desirably, the microcatheter has progressive pitch towards the distal tip—in other words, the distance between cuts gets smaller towards the distal tip. The pitch desirably is between about 0.05 and 0.10 C/mm at the proximal end of the tip relief region and between about 30 and 60 C/mm at the distal tip, desirably between about 0.15 (proximal end of tip relief region) and 50 C/mm (distal end of tip relief region).

The angle of the cuts can vary between about 20° to 89°.

The total length of the tip relief region is between about 10 mm and 100 cm, desirably between about 1 and 30 cm, more desirably between about 12 and 20 cm, and more desirably between about 12 and 15 cm for neurological applications. For use in a microcatheter assembly, the length of the tip relief desirably is about the same length as the “floppy” distal segment of the second catheter.

The length of the microcatheter can vary between about 60 to 200 cm, desirably between about 120 and 180 cm.

The distal end cut, having variable pitch, makes the microcatheter more kink-resistant and extraordinarily flexible (due to the fine pitch at the distal end). Because the proximal end region is not cut, the microcatheter has secure handling and superior pushability, even in tortuous vessels.

The microcatheter can be made of the same material its entire length, or can be made of joined together segments made of different materials.

The microcatheter can be coated along its full length if desired to promote lubriciousness and biocompatibility. The prior art contains many examples of coatings that can be used as well as methods for coating.

If full thickness cuts are made, the tip relief region can be coated to seal the perforations formed by the cuts, if desired. A polyurethane elastomer is desirable based on mechanical properties, biocompatibility, and ease of application. Other materials can be used that provide the necessary biocompatibility and mechanical properties.

In one method of applying the coating, an appropriately sized mandrel is placed inside the microcatheter to provide support and void. The microcatheter is then immersed into a solvent containing the dispersed coating material. Several dip-coat repetitions may be required to provide a leak-free and uniform barrier film. The coating may require exposure to elevated temperatures to aid in volatilizing the remaining solvent. Upon final cure, the mandrel is removed.

The coating could alternatively be formed by bonding an extruded polymer tube to the microcatheter using solvent bonding techniques or epoxy bonding techniques.

A preferred method of coating the microcatheter is by shrink wrapping. A tubular sleeve is placed over the microcatheter (either just the tip relief region or a larger portion of the microcatheter) and then heated to shrink. Any biocompatible heat shrink material can be used. Desirable materials are polyethylene terephthalate (PET), fluorinated ethylene-propylene (FEP), polyester (PE), and polytetrafluoroethylene (PTFE). Appropriate materials can be obtained from many commercial suppliers. A shrink ratio of about 0.1-9:1 is desirable.

Of course, alternate methods of coating can be used. It may be desirable to apply a coating of an additional low-friction, “slick” silicone or hydrophilic coating to the microcatheter, e.g. over the entire length or the tip relief region.

The proximal end of the microcatheter is desirably provided with an adaptor 20, which allows introduction of a liquid through the catheter. For example, the adaptor can be a luer lock adaptor which allows attachment of a syringe.

### Method of Making the Microcatheter

In one method of making the microcatheter, a medical grade superelastic nitinol tube (for example, nitinol BB-grade alloy—55.8% by wt. nickel/balance titanium—from Memry Corporation) is spiral cut using a CNC (Computer Numeric Controlled) laser. The cut is full thickness and progressive in pitch.

The tubing is attached to a machine with a predetermined tubing feed rate. A cutting element (such as a laser in this example) is placed across the tubing and the machine is activated to rotate and feed the tubing. As rotation of the machine (screw thread) occurs, the tubing moves axially and rotationally causing the tubing to be cut in a spiral manner by the laser. The machine can be set up to cut either a right or left hand spiral. The feed and speed rates can also be set to cut continuous or variable pitch spirals, or multizone spiral sections in which each zone has a unique pitch.

To seal the tip relief portion, an appropriately sized mandrel can be placed inside the microtube to provide support and improved heat transfer. The heat shrinkable tubing is advanced over the desired section of the microcatheter and heated using a heat gun or other thermal source. When cool, the mandrel is removed.

### II. Microcatheter Assembly

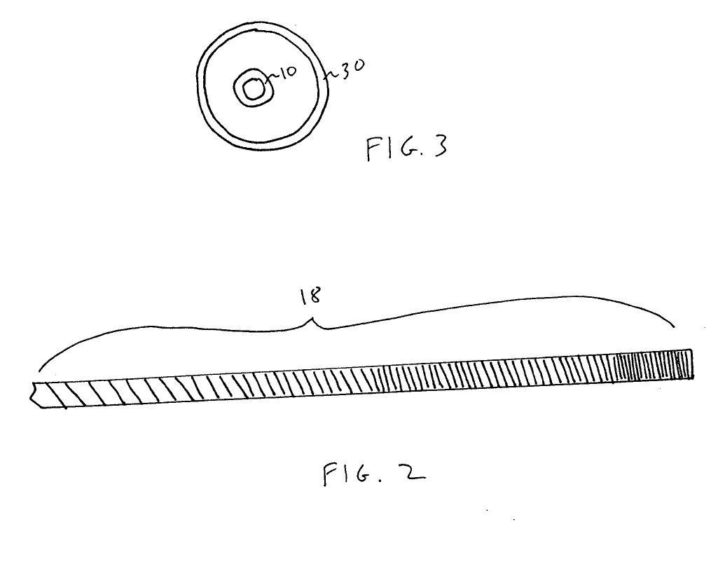

The microcatheter assembly is formed using the microcatheter described above and a larger diameter catheter (the second catheter), such as an infusion catheter. The second catheter can be one that is commercially available, such as a Tracker 18 or FasTracker 325. The second catheter should be of appropriate size to access the intended area. The microcatheter and the second catheter should be appropriately sized so that the microcatheter can be slidably inserted within the second catheter. FIG. 3 illustrates the microcatheter assembly cross-sectionally, where 10 is the microcatheter and 30 designates the second catheter.

In general, the inner diameter of the second catheter is dimensioned with respect to the outside diameter of the microcatheter to provide sufficient clearance to allow a liquid to pass through the second catheter. For a more viscous liquid, the clearance should be greater. The microcatheter should move easily within the second catheter in an axial direction. The inner diameter of the second catheter is desirably about 0.011 inches larger than the outer diameter of the microcatheter.

To form a coaxial microcatheter assembly below about 2.8 Fr, particularly suitable for neurovascular use, the second catheter is about 2.8 Fr or less and the microcatheter desirably has an inner diameter ranging from about 0.007 to 0.012 inches and an outer diameter ranging from about 0.010 to 0.018 inches.

FIG. 4 illustrates one embodiment of a microcatheter assembly. The assembly 40 includes second catheter 30, which is attached to the manifold 42 at its proximal end via a luer adaptor, for example. The manifold 42 includes a syringe adaptor 44 which provides connection (via a luer lock for example) between the interior space of the manifold 42 (which leads into the second catheter) and a syringe (not shown).

The manifold 42 includes a second adaptor 46 to receive the microcatheter 10. This can be a Tuohy-Borst adaptor, through which the microcatheter can be inserted. The microcatheter 10 is then pushed through the manifold and into and through the second catheter 30. A syringe (not shown) is fastened to the microcatheter 10 for delivery of a solution.

If desired, the two syringes are retained within a syringe holder (not shown) which allows synchronized delivery of the two solutions. The manifold would desirably be designed so that the syringes are aligned.

For placement of the catheters within the vasculature at the intended application site, the system can include a guidewire (not shown). A removable mandrel can be used to support the microcatheter during insertion of the microcatheter within the second catheter. It may be useful to provide a stop on the microcatheter to control the depth of its penetration into the second catheter.

Use with a Two-Part Prepolymer Composition

The microcatheter assembly can be used to deliver a two part prepolymer system used to form a hydrogel in situ. In one embodiment, each of the two parts includes one part of a redox couple. When the two parts are combined, crosslinking (formation of the hydrogel) begins. Premature mixing of the two parts can lead to unintended, premature formation of the hydrogel (and clogging of the catheter, for example). In one embodiment, the initiator solution (less viscous) is delivered through the microcatheter lumen and the prepolymer solution (more viscous) is delivered through the second catheter lumen. The viscosities of the prepolymer solution and initiator solution can vary and should be appropriate for the size catheter being used. Generally, for a catheter ranging from about 3 Fr to 8 Fr, a viscosity of about 10 to 200 cps is appropriate. For a catheter ranging from about 1.6 to 3 Fr, a viscosity ranging from about 1 to 40 is appropriate. The solution can theoretically be any viscosity so long as it can be transferred through the catheter.

Accordingly, the initiator solution delivered through the microcatheter 10 does not contact the prepolymer solution delivered through the second catheter 30. It may be desirable to position the microcatheter within the second catheter so that a mixing chamber is formed at the distal tips of the catheters. In other words, it may be desirable to position the microcatheter so that its tip is recessed from the distal tip of the second catheter.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

What is claimed is:

1. A microcatheter having an elongate tubular body, a distal portion, a proximal portion, and a tip relief region within the distal end portion, wherein the tip relief region comprises at least one spiral cut.

2. The microcatheter of claim 1, having a diameter below about 2.8 Fr.

3. The microcatheter of claim 1, having a diameter between about 2.8 Fr and 0.7 Fr.

4. The microcatheter of claim 1, wherein the microcatheter is made from a material selected from the group consisting of platinum, platinum alloys, nickel alloys, titanium alloys, stainless steel, polyimide, polyethylene, polyurethane, and PTFE.

5. The microcatheter of claim 1, wherein the microcatheter is made from medical grade superelastic nitinol.

6. The microcatheter of claim 1, wherein the spiral cuts are full thickness cuts.

7. The microcatheter of claim 1, wherein the cuts have a progressive pitch towards the distal tip.

8. The microcatheter of claim 1, wherein the pitch ranges between about 0.05 and 60 C/mm.

9. The microcatheter of claim 7, wherein the pitch is between about 0.05 and 0.10 C/mm at the proximal end of the tip relief region.

10. The microcatheter of claim 7, wherein the pitch is between about 30 and 60 C/mm at the distal end of the tip relief region.

11. The microcatheter of claim 1, wherein the length of the tip relief region is between about 1 and 30 cm.

12. The microcatheter of claim 1, wherein the tip relief region is coated.

13. The microcatheter of claim 12, wherein the tip relief portion is coated by shrink wrapping.

14. A coaxial dual lumen microcatheter assembly formed by inserting the microcatheter of claim 1 coaxially within a second catheter.

15. A method for forming a hydrogel in situ at a desired site in a body, comprising the steps:
providing a prepolymer composition comprising first and second solutions that will form a hydrogel when mixed;
providing a first catheter having an elongate tubular body, a distal portion, a proximal portion, and a tip relief region within the distal end portion, wherein the tip relief region comprises one or more spiral cuts having a progressive pitch towards the distal end of the tip relief region, and wherein the pitch is between about 30 and 60 C/mm at the distal end of the tip relief region;
providing a second catheter having proximal and distal ends and a lumen large enough to accept the first catheter;
assembling the first and second catheters into a coaxial dual lumen assembly, so that the distal end of the second catheter extends further than the distal end of the first catheter and a mixing chamber is formed at the distal end of the assembly;
delivering the first and second catheters to the body so that the distal ends of the catheters are at the intended site for hydrogel formation; and
delivering the first or second solution through the first catheter and the other solution through the second catheter, so that the solutions mix in the mixing chamber and form the hydrogel upon delivery.

16. The method of claim 15, wherein the first catheter has a diameter below about 2.8 Fr.

17. The method of claim 15, wherein the first catheter has a diameter between about 2.8 Fr and 0.7 Fr and is made from medical grade superelastic nitinol.

18. The method of claim 15, wherein the spiral cuts on the first catheter are full thickness cuts.

19. The method of claim 15, wherein the pitch of the one or more spiral cuts on the tip relief region of the first catheter ranges between about 0.05 and 60 C/mm.

20. The method of claim 15, wherein the pitch is between about 0.05 and 0.10 C/mm at the proximal end of the tip relief region.

21. The method of claim 15, wherein the length of the tip relief region on the first catheter is between about 1 and 30 cm.

22. The method of claim 15, wherein the tip relief region on the first catheter is coated.

23. The method of claim 22, wherein the tip relief region on the first catheter is coated by shrink wrapping.

24. The method of claim 15, wherein the first and second solutions of the prepolymer composition each contains one part of a redox couple and at least one of the first or second solutions contains the prepolymer.
